(19) United States
(12) Patent Application Publication
(10) Pub. No.: US 2009/0203968 A1
(43) Pub. Date: Aug. 13, 2009

(54) VAGINAL SPECULUM WITH SOLID ROUNDED EDGES

(76) Inventor: Kevin Winslow, Jacksonville, FL (US)

Correspondence Address: MARK YOUNG, P.A., 12086 FORT CAROLINE ROAD, UNIT 202, JACKSONVILLE, FL, 32225, US

(21) Appl. No.: 12/027,273

(22) Filed: Feb. 7, 2008

(51) Int. Cl. A61B 1/32

(52) U.S. Cl. 600/220

(57) ABSTRACT

A vaginal speculum includes a pair of opposed blades. One of the opposed blades is movably mounted for controllable movement towards and away from the other of the pair of opposed blades. Each blade includes an elongate arcuate paddle defining an interior compartment between the paddles. A solid rounded lip defines the terminal end of each side and the distal end of the blades. The lips may comprise a unitary structure which may be integrally formed with the paddle or separately formed and attached to each paddle such as by welding or mechanical attachment. The solid rounded lips protrude laterally from the first and second sides and the distal end of the blade to provide a smooth brim configured to urge tissue away from the blade. The solid rounded lip is preferably flush with the interior compartment defined by each elongate arcuate paddle. The solid rounded lip has a major radius of at least 2 mm and preferably 2 mm to 5 mm and approximately ½ to 1/20 times the blade radius.

### FIELD OF THE INVENTION

This invention generally relates to a vaginal speculum used for inspection of the vagina and cervix, and, more particularly, to a speculum having a solid rounded lip forming the free edge of each speculum blade.

### BACKGROUND

A vaginal speculum is a diagnostic instrument for dilating the opening of the vagina cavity to facilitate access for observation and treatment. A typical prior art vaginal speculum 100, as shown in FIGS. 1 and 2, has a pair of blades 110, 145, each having a curved distal end 105, 150. The blades 110, 145 are inserted into the vagina in a closed state, with the distal ends 105, 150 initiating penetration into the vagina. When the blades 110, 145 are fully inserted the handles 125, 140 are squeezed towards one another, causing one blade (i.e., the upper blade 110 in the example speculum shown in FIGS. 1 and 2) to pivot away from the opposite blade (i.e., the lower blade 145 in the example speculum shown in FIGS. 1 and 2). The pivoting movement of the blade 110 dilates the vaginal cavity. An adjustment fork 135 and threaded locking nut 130 are provided to enable vertical adjustment of the distance between the pivoting axis of the upper blade 110 and the pivoting axis of the lower blade 145. Thus, the speculum 100 may be adjusted to work within narrow and wide vaginal orifices, such as openings widened due to loss of muscle tone as a result of age or previous childbirths. A threaded locking shaft 120 and nut 115 are also provided to hold the upper blade 110 in an opened position within a dilated vaginal cavity.

Unfortunately, patients frequently complain that conventional vaginal specula cause extreme discomfort. Such devices are typically comprised of surgical steel or like material, which can be sterilized in an autoclave before use. Such materials are initially unpleasantly cold to the touch.

Dilation of the vaginal orifice and cavity with a conventional surgical steel speculum can be quite painful. Even when the blades 110, 145 are in a closed position, portions of the free edges 155 are exposed. The free edges 155 of the blades 110, 145 tend to be thin and feature sharp right angle, bevelled or chamfered corners. As the speculum blades 110, 145 are urged into a vaginal cavity, tissue that projects against the free edges 155 is scraped. As the blades 110, 145 are pivoted to dilate the vaginal cavity, tissue projected against the exposed free edges is grated. As the opening of the vagina has a circular muscle that is more resistant to dilations than the vaginal cavity, during withdrawal the speculum blades must be collapsed. Pivotal movement of the blades towards each other pinches protruding tissue. This scraping and pinching cause discomfort as well as trauma to the vaginal tissue.

What is needed is an improved speculum that prevents or minimizes scraping and pinching of vaginal tissue caused by conventional specula. The improved speculum should be compatible with surgical steel or like materials for durability and to permit sterilization using conventional techniques (e.g., in an autoclave). The invention is directed to overcoming one or more of the problems and solving one or more of the needs as set forth above.

### SUMMARY OF THE INVENTION

To solve one or more of the problems set forth above, in an exemplary implementation of the invention, a speculum having a rounded lip forming the free edge of each speculum blade is provided. The vaginal speculum includes a pair of opposed blades configured for insertion into a vaginal orifice. One of the opposed blades is movably mounted for controllable movement towards and away from the other of the pair of opposed blades. Each blade includes an elongate arcuate paddle defining an interior compartment. The elongate arcuate paddle has first and second sides, a proximal end and a distal end. Each of the first and second sides, proximal end and distal end have a terminal end. Each blade further includes a solid rounded lip defining the terminal end of each of the first and second sides. Each blade further includes a solid rounded lip defining the terminal end of the distal end. The movably mounted opposed blade is pivotally mounted for pivoting movement towards and away from the other of the pair of opposed blades. The distal end of each blade comprises a spheroid quadrant formed integrally with the elongate arcuate paddle. The lips may comprise a unitary structure which may be integrally formed with the paddle or separately formed and attached to each paddle such as by welding or mechanical attachment. The solid rounded lips protruding laterally from the first and second sides and the distal end of the blade to provide a smooth brim configured to urge tissue away from the blade. The solid rounded lip is preferably flush with the interior compartment defined by each elongate arcuate paddle. The solid rounded lip has a major radius of at least 2 mm and preferably 2 mm to 5 mm and approximately ½ to 1/20 times the blade radius, which is large enough to comfortably distribute stresses on contacted vaginal tissue. The speculum is comprised of surgical stainless steel, or other metal such as martensitic steel, austenitic steel, titanium, and titanium alloy, or a plastic such as polysulphone, polyetherimide, polyphenylsulfone, polyphenylene sulfide, polyetheretherketone, and syndiotactic polystyrene. The material is capable of withstanding repeated sterilization in an autoclave.

The exemplary speculum features blades with solid rounded lips extending outwardly from the free edges of the blades. Advantageously, each lip is a solid structure that prevents infiltration by contaminants and entrapment of tissue. Each lip is comprised of a material that can be sterilized in an autoclave or using other conventional sterilization techniques, if the device is reusable. Each lip protrudes laterally from the side edges and distal end of the blade body to provide a smooth brim that urges adjacent tissue away from the blade, without encroaching into or comprising visibility through the interior compartment defined by the space between the blades.

### BRIEF DESCRIPTION OF THE DRAWINGS

The foregoing and other aspects, objects, features and advantages of the invention will become better understood with reference to the following description, appended claims, and accompanying drawings, where:

FIG. 1 is a side view of an exemplary prior art speculum; and

FIG. 2 is a perspective view of the exemplary prior art speculum; and

FIG. 3 shows a perspective view of an exemplary upper blade of an exemplary speculum according to principles of the invention; and

FIG. 4 shows a perspective view of an exemplary lower blade of an exemplary speculum according to principles of the invention; and

FIG. 5 shows a side view of an exemplary speculum according to principles of the invention; and

FIG. 6 shows a perspective view of an exemplary speculum according to principles of the invention; and

FIG. 7A shows a profile view of a section of an exemplary blade of a speculum according to principles of the invention; and

FIG. 7B shows a perspective view of a section of an exemplary blade of a speculum according to principles of the invention; and

FIG. 8A shows a profile view of a section of an exemplary blade of a speculum according to principles of the invention; and

FIG. 8B shows a perspective view of a section of an exemplary blade of a speculum according to principles of the invention; and

FIG. 9A shows a profile view of a section of an exemplary blade of a speculum according to principles of the invention; and

FIG. 9B shows a perspective view of a section of an exemplary blade of a speculum according to principles of the invention; and

FIG. 10A shows a perspective view of a section of an exemplary rounded lip of a speculum according to principles of the invention; and

FIG. 10B shows a plan view of a section of an exemplary rounded lip of a speculum according to principles of the invention; and

FIG. 11A shows a close-up plan view of a rounded lip of a speculum according to principles of the invention; and

FIG. 11B shows a close-up plan view of another rounded lip of a speculum according to principles of the invention; and

FIG. 12A shows a profile view of a section of an exemplary rounded lip of a speculum according to principles of the invention; and

FIG. 12B shows a perspective view of a section of an exemplary rounded lip of a speculum according to principles of the invention; and

FIG. 13A shows a profile view of a section of an exemplary rounded lip of a speculum according to principles of the invention; and

FIG. 13B shows a perspective view of a section of an exemplary rounded lip of a speculum according to principles of the invention.

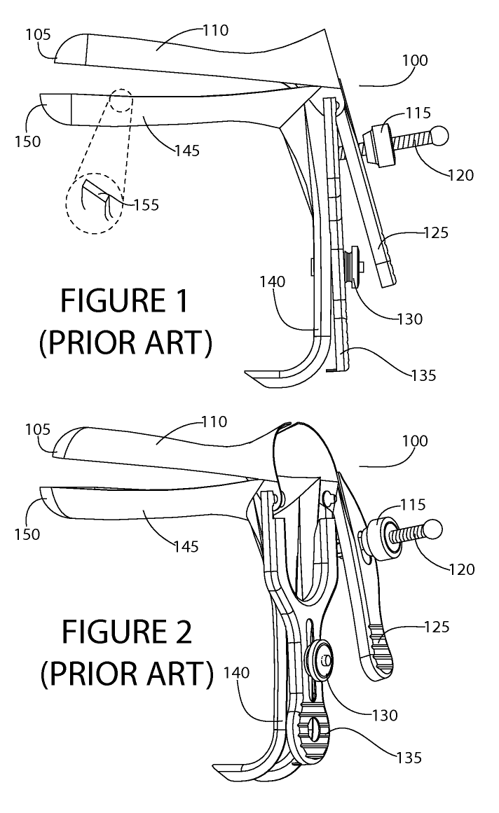

Those skilled in the art will appreciate that the figures are not intended to be drawn to any particular scale; nor are the figures intended to illustrate every embodiment of the invention. The invention is not limited to the exemplary embodiments depicted in the figures or the speculum, shapes, relative sizes, ornamental aspects or proportions shown in the figures.

### DETAILED DESCRIPTION

Referring to the Figures, in which like parts are indicated with the same reference numerals, various views of an exemplary speculum and components thereof according to principles of the invention are shown. The term “speculum” is used herein for convenience to refer to any vaginal speculum with a pair of opposed blades, regardless of the orientation of the blades. The invention is not limited to the particular exemplary speculum shown in the Figures. Instead, the invention may be utilized with any type of vaginal speculum with a pair of opposed blades, in any sizes.

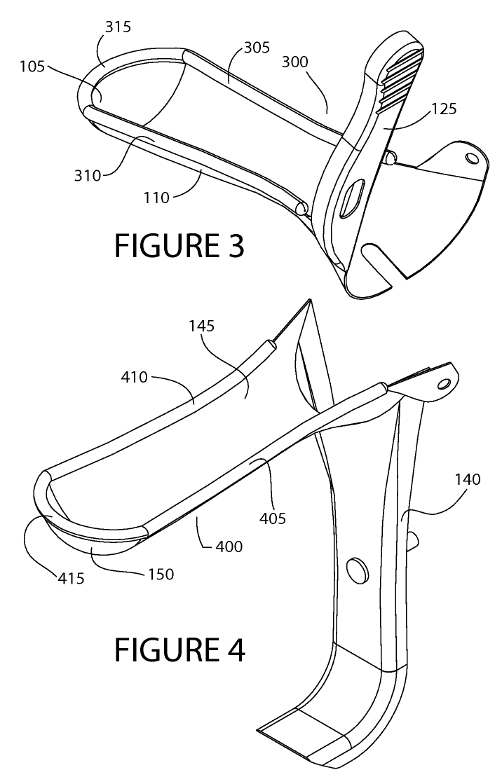

Referring now to FIG. 3, a perspective view of an exemplary upper blade 300 is conceptually illustrated. The upper blade 300 has a generally L-shaped configuration with a blade body 110 configured (i.e., sized and shaped) for insertion into a vaginal orifice, and a handle 125 substantially perpendicular to the blade body 110. The blade body 110 has a semicircular or concave cross-section. The distal end 105 of the blade 300, i.e., the end opposite to the handle 125, is a spherical (or nearly spherical) quadrant 105 formed integrally with the remaining body of the blade 110.

A smooth rounded lip 305, 310, 315 covers the free edge of each side of the blade 300, including the free edge of the distal end 105. The rounded lip 305, 310, 315 may be a separate structure attached to or formed integrally with the blade body 110 and distal end 105. The rounded lip 305, 310, 315 protrudes laterally from the edge of the blade body 110 and distal end 105 to provide a smooth brim that urges adjacent tissue away from the blade 300. The lip is preferably solid, as a hollow or rolled structure is conducive to infiltration by contaminants and tissue and entrapping contaminants in the compartment of the hollow or rolled structure.

Referring now to FIG. 4, a perspective view of an exemplary lower blade 400 is conceptually illustrated. Like the upper blade 300, the lower blade 400 has a generally L-shaped configuration with a blade body 145 configured (i.e., sized and shaped) for insertion into a vaginal orifice, and a handle 140 substantially perpendicular to the blade body 145. The blade body 145 has a semicircular or concave cross-section. The distal end 150 of the blade 400, i.e., the end opposite to the handle 140, is a spherical (or nearly spherical) quadrant 150 formed integrally with the remaining body of the blade 145. The lower blade 400 is longer than the upper blade 300, enabling the distal end 150 of the lower blade 400 to extend beneath the ectocervix.

As with the upper blade 300, a smooth rounded lip 405, 410, 415 covers the free edge of each side of the blade 400, including the free edge of the distal end 150. The rounded lip 405, 410, 415 may be a separate structure attached to or formed integrally with the blade body 145 and distal end 150. The rounded lip 405, 410, 415 protrudes laterally from the edge of the blade body 145 and distal end 150 to provide a smooth brim that urges adjacent tissue away from the blade 400.

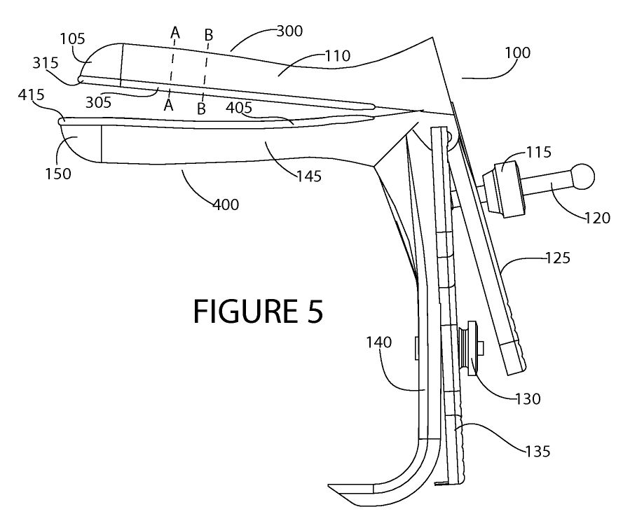

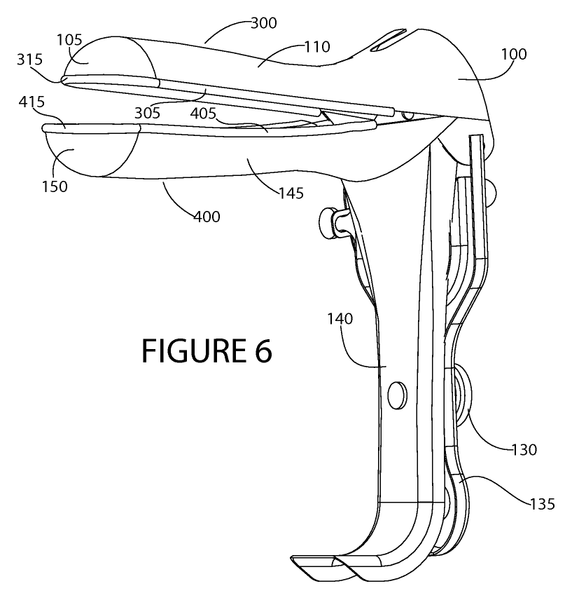

Referring to FIGS. 5 and 6, side and perspective views of an exemplary vaginal speculum 100 having a pair of blades 300, 400 equipped with rounded lips is conceptually shown. The blades 300, 400 are configured for insertion into a vaginal orifice in a closed state, with the distal ends 105, 150 initiating penetration into the vagina. When the blades 300, 400 are fully inserted, the handles 125, 140 are squeezed towards one another, causing one blade (i.e., the upper blade 300 in the example speculum shown in FIGS. 5 and 6) to pivot away from the opposite blade (i.e., the lower blade 400 in the example speculum shown in FIGS. 5 and 6). The pivoting movement of the blade 300 dilates the vaginal cavity. An adjustment fork 135 and threaded locking nut 130 are provided to enable vertical adjustment of the distance between the pivoting axis of the upper blade 300 and the pivoting axis of the lower blade 400. Thus, the speculum 100 may be adjusted to work within narrow and wide vaginal orifices, such as openings widened due to loss of muscle tone as a result of age or previous childbirths. A threaded locking shaft 120 and nut 115 are also provided to hold the upper blade 300 in an opened position within a dilated vaginal cavity.

As discussed above, a smooth rounded lip 305, 310, 315 covers the free edge of each side of the blade 300, including the free edge of the distal end 105. The rounded lip 305, 310, 315 may be a separate structure attached to or formed integrally with the blade body 110 and distal end 105. The rounded lip 305, 310, 315 protrudes laterally from the edge of the blade body 110 and distal end 105 to provide a smooth brim that urges adjacent tissue away from the blade 300.

As with the upper blade 300, a smooth rounded lip 405, 410, 415 covers the free edge of each side of the blade 400, including the free edge of the distal end 150. The rounded lip 405, 410, 415 may be a separate structure attached to or formed integrally with the blade body 145 and distal end 150. The rounded lip 405, 410, 415 protrudes laterally from the edge of the blade body 145 and distal end 150 to provide a smooth brim that urges adjacent tissue away from the blade 400.

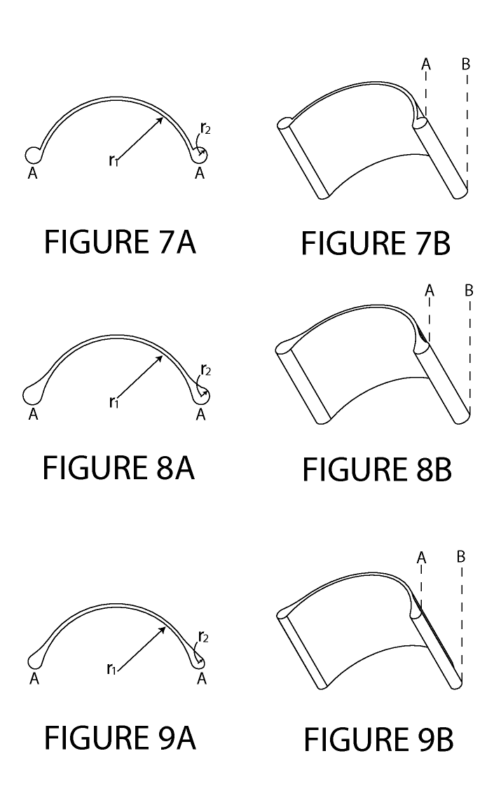

The upper and lower blades 300, 400 have similar or identical cross-section shapes. Illustratively, exemplary embodiments of section A-B of the exemplary lower blade 400 are shown in FIGS. 7A, 7B, and FIGS. 8A, 8B, and FIGS. 9A, 9B. In each example, the rounded lip does not protrude substantially, if at all, into the interior compartment defined by the blade. Thus, the lip does not impede visibility through the interior compartment. In each example, the rounded portion of the lip protrudes outwardly in a lateral direction from the blade. The outward rounded protrusion comprises a smooth brim that urges tissue away from the edge of the blade 400. The major radius r₂ (i.e., the largest most prominent radius) of the protrusion is approximately at least 2 mm, which provides a broad enough surface to comfortably distribute force exerted on abutting tissue, avoid stress concentrations, and maintain a safe distance between the tissue and the interior compartment defined by the blades. A major radius r₂ of approximately 2 mm to 5 mm is preferred. The radius r₁ of the blades is approximately 10 mm to 20 mm. Thus, the preferred ratio of the blade radius r₁ to the protrusion radius r₂ is approximately 2 to 10. In the embodiments illustrated in FIGS. 8A, 8B and FIGS. 9A, 9B, the outwardly projecting portion of the lip eases into a convex curve that gradually merges with the blade, forming a teardrop shape.

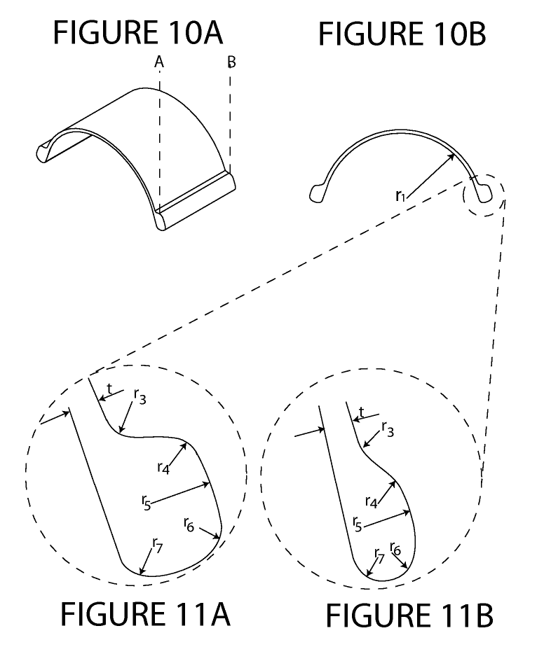

Referring now to FIGS. 10A, 10B, 11A and 11B, embodiments with rounded lips having broad lateral edges, i.e., a flat or substantially flat lateral edge or a lateral edge with a relatively large radius of curvature r₅, and a relatively small superior lateral radius r₄ and a relatively small inferior lateral radius r₆. Superior and inferior medial radii r₃ and r₇ provide a smooth gradual curved transition from the lip to the blade. The lip profile (i.e., a section plan view as illustrated in FIGS. 11A and 11B) generally has a shape of a rectangle with rounded corners or a superellipse, Lamé curve, squircle, filleted square or filleted rectangle. Other profiles resembling a circle, oval, ellipse, ellipsoid, hyperellipse, hypoellipse, parabola, may also be utilized and are intended to come within the scope of the invention. The rounded lip does not protrude substantially, if at all, into the interior compartment defined by the blade. Thus, the lip does not impede visibility through the interior compartment. In each example, the lip protrudes outwardly in a lateral direction from the blade. The outward protrusion comprises a smooth brim with a broad lateral edge that distributes stress, avoids stress concentrations, and urges tissue away from the edge of the blade 400. The major radius r₅ (i.e., the largest most prominent radius) of the protrusion is approximately at least 2 mm, which provides a broad enough surface to comfortably distribute force exerted on abutting tissue, avoid stress concentrations, and maintain a safe distance between the tissue and the interior compartment defined by the blades. The major radius r₅ is approximately at least twice the thickness t of the blade, and preferably approximately 2 to 5 times the thickness t of the blade. A major radius r₂ of approximately 2 mm to 5 mm is preferred. The other lateral radii r₄ and r₆ are smaller than the major radius. Lateral and medial transitional radii r₃ and r₇ are configured to provide a smooth, gradual, curved transition from the rounded lip to the blade. In the embodiments illustrated in FIGS. 10A, 10B and FIGS. 11A, 11B, the outwardly projecting portion of the lip eases into a convex curve that gradually merges with the blade.

The lips 400, 500 may be integrally formed with the blades 110, 145 or separately formed and attached to the blades 110, 145. Blades 110, 145 with integrally formed lips may be produced by machining or casting. Rounded edges may be formed by folding blade material. In a preferred embodiment, the folded sections are tightly packed to avoid formation of any cavities or compartments that may otherwise entrap contaminants.

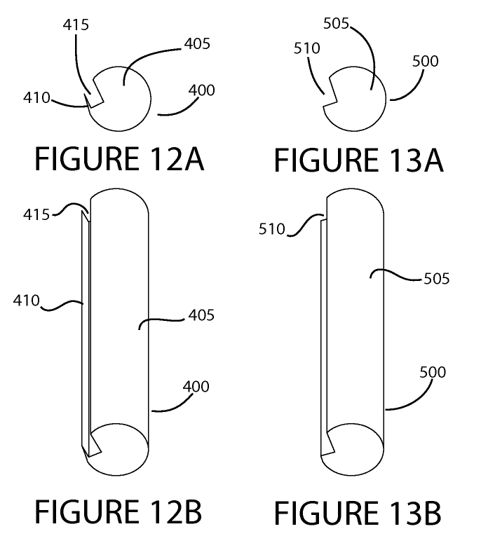

Separately formed lips 400, 500 may be surface mounted to edges of the speculum blades 110, 145. Alternatively, the separately formed lips 400, 500, sections of which are conceptually illustrated in FIGS. 12A, 12B, 13A and 13B, may include a channel 415 or cutout 510 to receive the free edge of a blade 110, 145. The channel 415 or cutout 510 may be sized and shaped to securely receive the free edge. In a preferred implementation, the channel or cutout is off-center relative to the cross-section of the lip. This eccentric arrangement positions the edge of the blade flush (as in FIGS. 13A, 13B) or nearly flush with a side of the lip. A transition 410 may be provided to smoothly merge with the free edge of the blade. Thus, the rounded lip does not protrude substantially, if at all, into the interior compartment defined by the blade. Consequently, the lip does not impede visibility through the interior compartment formed by the blade. Concomitantly, the rounded portion 405, 505 of the lip 400, 500 protrudes outwardly in a lateral direction from the blade. The outward rounded protrusion comprises a smooth brim that urges tissue away from the edge of the blade 110, 145, minimizes or eliminates sharp edges that contact adjacent tissue and reduces and/or eliminates pinching. The rounded protrusion also facilitates insertion of the speculum blades into the vaginal cavity. The invention is not limited to the attachable circular projection illustrated in FIGS. 12A, 12B, 13A and 13B. Other rounded configurations, including but not limited to the teardrop shapes, of FIGS. 8A, 8B, 9A and 9B, may comprise attachable lips in accordance with principles of the invention.

The lips and blades may be comprised of the same materials. In a preferred embodiment, the composition is stainless steel, particularly surgical stainless steel, which is easy to clean and sterilize, strong and corrosion-resistant. However, the invention is not limited to a particular material. Other materials suitable for medical device manufacture may be used within the scope of the invention. For example, other metals such as other martensitic steels, austenitic steels, titanium and alloys thereof may be utilized. Additionally, medical equipment grade plastics may be used in disposable and reusable embodiments. By way of example and not limitation, polysulphone, polyetherimide, polyphenylsulfone, polyphenylene sulfide, polyetheretherketone, and syndiotactic polystyrene, are plastic materials of choice for implementations that can be autoclaved.

In embodiments where the lips 400, 500 are separately formed and attached to the blades 110, 145, welding, soldering, brazing and mechanical attachments may be utilized. Welding entails coalescence by melting the work pieces (i.e., the blades and lips) and adding a filler material to form a pool of molten material (i.e., a weld puddle) that cools to become a strong joint. Soldering and brazing involve melting a lower-melting-point material between the work pieces to form a bond between them, without melting the work pieces. Attachment comprising mechanical joints, such as interlocking tongue and groove joints may also be used.

In embodiments where the lips 400, 500 are separately formed and attached to the blades 110, 145, casting and machining operations may be utilized. Machining operations include turning, milling, drilling, and grinding operations.

Advantageously, regardless of the method of manufacture, the resulting speculum features blades with rounded lips extending outwardly from the free edges of the blades. Each lip is a solid structure that prevents infiltration by contaminants and entrapment of tissue. Each lip is comprised of a material that can be sterilized in an autoclave or using other conventional sterilization techniques, if the device is reusable. Each lip protrudes laterally from the side edges and distal end of the blade body to provide a smooth brim that urges adjacent tissue away from the blade.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A vaginal speculum comprising a pair of opposed blades configured for insertion into a vaginal orifice, one of said opposed blades being movably mounted for controllable movement towards and away from the other of the pair of opposed blades, each blade comprising an elongate arcuate paddle defining an interior compartment, said elongate arcuate paddle having first and second sides, a proximal end and a distal end, each of the first and second sides, proximal end and distal end having a terminal end, each blade further comprising a solid rounded lip defining the terminal end of each of the first and second sides.

2. A vaginal speculum according to claim 1, said solid rounded lip having a cross-sectional profile from the group consisting of a rounded rectangle, a superellipse, Lamé curve, squircle, circle, oval, ellipse, hyperellipse, hypoellipse, parabola and teardrop.

3. A vaginal speculum according to claim 1, each blade further comprising a solid rounded lip defining the terminal end of the distal end.

4. A vaginal speculum according to claim 1, wherein one of said opposed blades is pivotally mounted for pivoting movement towards and away from the other of the pair of opposed blades.

5. A vaginal speculum according to claim 1, the distal end of each blade comprises a spheroid quadrant formed integrally with the elongate arcuate paddle comprising the blade.

6. A vaginal speculum according to claim 1, each blade further comprising a solid rounded lip defining the terminal end of the distal end, and said solid rounded lip defining the terminal end of each of the first and second sides and the distal end comprising a unitary structure.

7. A vaginal speculum according to claim 1, each blade further comprising a solid rounded lip defining the terminal end of the distal end, and said solid rounded lip defining the terminal end of each of the first and second sides and the distal end comprising a structure integrally formed with each blade.

8. A vaginal speculum according to claim 1, each blade further comprising a solid rounded lip defining the terminal end of the distal end, and said solid rounded lip defining the terminal end of each of the first and second sides and the distal end comprising a unitary structure attached to each paddle.

9. A vaginal speculum according to claim 1, each blade further comprising a solid rounded lip defining the terminal end of the distal end, and said solid rounded lip defining the terminal end of each of the first and second sides and the distal end comprising a unitary structure attached by welding to each paddle.

10. A vaginal speculum according to claim 1, each blade further comprising a solid rounded lip defining the terminal end of the distal end, and said solid rounded lip defining the terminal end of each of the first and second sides and the distal end being attached to each paddle by a mechanical joint.

11. A vaginal speculum according to claim 1, each blade further comprising a solid rounded lip defining the terminal end of the distal end, and said solid rounded lip protruding laterally from the first and second sides and the distal end of the blade.

12. A vaginal speculum according to claim 1, each blade further comprising a solid rounded lip defining the terminal end of the distal end, and said solid rounded lip protruding laterally from the first and second sides and the distal end of the blade comprising a smooth brim configured to urge tissue away from the blade.

13. A vaginal speculum according to claim 1, each blade further comprising a solid rounded lip defining the terminal end of the distal end, and said solid rounded lip protruding laterally from the first and second sides and the distal end of the blade, said solid rounded lip further being flush with the interior compartment defined by each elongate arcuate paddle.

14. A vaginal speculum according to claim 1, each blade further comprising a solid rounded lip defining the terminal end of the distal end, and said solid rounded lip protruding laterally from the first and second sides and the distal end of the blade, said solid rounded lip having a major radius of at least 2 mm.

15. A vaginal speculum according to claim 1, each blade further comprising a solid rounded lip defining the terminal end of the distal end, and said solid rounded lip protruding laterally from the first and second sides and the distal end of the blade, said solid rounded lip having a major radius of 2 mm to 5 mm.

16. A vaginal speculum according to claim 1, each blade further comprising a solid rounded lip defining the terminal end of the distal end, and said solid rounded lip protruding laterally from the first and second sides and the distal end of the blade, said blades each having a blade radius defining the arcuate paddle, and said solid rounded lip having a major radius of approximately ½ to 1/20 times the blade radius.

17. A vaginal speculum according to claim 1, each solid rounded lip having a substantially flat lateral edge, a smaller superior lateral radius and inferior lateral radius, and superior and inferior medial radii that provide a smooth gradual curved transition from the solid rounded lip to the blade.

18. A vaginal speculum according to claim 1, each solid rounded lip having a lateral edge with a first radius of curvature, a smaller superior lateral radius and inferior lateral radius, and superior and inferior medial radii that provide a smooth gradual curved transition from the solid rounded lip to the blade.

19. A vaginal speculum according to claim 1, said speculum being comprised of a plastic from the group consisting of polysulphone, polyetherimide, polyphenylsulfone, polyphenylene sulfide, polyetheretherketone, and syndiotactic polystyrene.

20. A vaginal speculum according to claim 1, each blade further comprising a solid rounded lip defining the terminal end of the distal end, and the speculum being comprised of a material that can withstand repeated sterilization in an autoclave.
